(19) United States
(12) Patent Application Publication
(10) Pub. No.: US 2002/0151599 A1
(43) Pub. Date: Oct. 17, 2002

(54) FORMULATIONS HAVING AN ANTIVIRAL ACTION

(76) Inventors: HERWIG BUCHHOLZ, FRANKFURT (DE); ANNETTE WAGNER, FRANKFURT (DE); CHRISTINE KRAUS, FRANKFURT (DE); JERZEY D. MEDUSKI, DARMSTADT (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/349,713

(22) Filed: Jul. 8, 1999

(51) Int. Cl. A61K 7/00; A61K 9/00; A61K 7/42; A61K 6/00; A61K 31/12

(52) U.S. Cl. 514/685; 424/59; 424/47

(57) ABSTRACT

The present invention relates to formulations in solid or liquid form comprising isoquercitrin as a natural flavonoid, which is present therein as a light protection filter and/or antiviral substance. The invention relates to both cosmetic and medicinal formulations.

The present invention relates to formulations in solid or liquid form comprising isoquercitrin as a natural flavonoid, which is present therein as a light protection filter and/or antiviral substance. The invention relates to both cosmetic and medicinal formulations.

As is known, the skin is sensitive to solar rays, which can cause simple sunburn or an erythema, but also burns of varying severity.

However, solar rays also have other adverse effects: they cause the skin to lose its elasticity and form wrinkles and thus lead to premature ageing. Dermatoses may also sometimes be observed. In extreme cases, some people develop skin cancer. In addition, it is known that people who suffer from herpes under stress in many cases likewise get herpes blisters on the lips after intensive solar irradiation.

As is known, the most dangerous solar rays are the ultraviolet rays having a wavelength of less than 400 nm. It is also known that the presence in the earth's atmosphere of the ozone layer, which absorbs some solar radiation, means that the lower limit of the ultraviolet rays which reach the earth's surface is approximately 280 nm.

It is thus desirable to provide both medicinal and cosmetic formulations which, on the one hand, protect the skin from the damaging influence of UV radiation, but, on the other hand, also have an antiviral action and thus, in cases where the herpes infection is present, are able to prevent the outbreak of an attack of herpes, which may be triggered by solar radiation. In view of a change in consumer behaviour, it would appear desirable to solve this problem as far as possible using environmentally compatible components. In particular, it is desirable to use naturally occurring substances for this purpose.

The object of the present invention is thus to provide cosmetic and/or medicinal preparations which comprise at least one naturally occurring light protection filter which can absorb UV rays in a wavelength range from 280 to 400 nm, i.e. UVB rays having a wavelength between 280 and 320 nm, which play a decisive role in the formation of a solar erythema, and also UVA rays having a wavelength between 320 and 400 nm, which cause the skin to tan, but also to age, encourage an erythematous reaction or heighten this reaction in certain people, or can even trigger phototoxic or photoallergic reactions or attacks of herpes. The object of the present invention is thus also to provide formulations which have an antiviral action against Herpesviridae, making them useful both for prophylaxis against the formation of herpetic disorders of the skin and also for the treatment of corresponding disorders.

The sunscreen filters commonly used in cosmetics today are divided into UVA and UVB filters. For both UV ranges, there are many tried and tested compounds known from the specialist literature, compounds cited here merely by way of example being those such as phenylbenzimidazole-5-sulfonic acid (Eusolex® 232), benzophenone derivatives (Eusolex® 4360), benzoylmethane or dibenzoylmethane derivatives (Eusolex® 9020 or Parsol® 1789, Eusolex 8020), triazone derivatives (Uvinul T 150®), salicylate derivatives (Eusolex HMS®, Eusolex OS®), benzylidenecamphor derivatives (Eusolex® 6300), octocrylene (Eusolex OCR®), inorganic light protection filters from the group of titanium dioxide and zinc oxide.

The present object is achieved by cosmetic or medicinal formulations comprising isoquercitrin.

Such formulations have an antiviral action, in particular against Herpesviridae, e.g., alphapherpesvirinae (herpes simplex-like viruses), simplex virus (herpes simplex-like viruses), variacellovirus (varicella-zoster virus). See also White and Fenner, Medical Virology, AP Press, 1994, Chapter 2. At the same time, the object is also achieved by cosmetic or medicinal formations comprising isoquercitrin as a UV filter having an antiviral action.

The invention provides cosmetic or medicinal formulations comprising isoquercitrin in the form of a cream, milk, lotion, oil, stick or spray for application to the skin, in the form of a mouth, nasal or inhalation spray, or in the form of tablets, dragées, capsules, syrup, juice or drops.

Formulations according to the invention may comprise isoquercitrin in combination with one or more UVA filters and/or one or more UVB filters.

In such formulations, UVA filters, selected from the group consisting of the benzophenones, benzoylmethane derivates and dibenzoylmethane derivates, may be present. The UVA filters also include those which are available commercially under the names Eusolex® 4360, Eusolex® 8020 or Eusolex® 9020. UVB filters present according to the invention may be selected from the group of cinnamic acid esters, such as isoamyl methaxycinnamate or octyl methoxycinnamate, triazines (e.g. Uvinul® T150), benzylidenecamphor (Eusolex® 6900), PABA (Eusolex 6007), octocrylene (Eusolex® OCR), salicylates (OS, HMS) and inorganic light protection filters, such as ZnO and and TiO₂. These light protection filters may be present individually or in combinations of different light protection filters. Light protection filter can protect against radiation from visible light in the range up to 800 mm.

The invention also provides those formulations in which the antiviral action of isoquercitrin is enhanced through combination with substances from the group consisting of 5-ethydeoxyuridine, quercetin, galangin, kaempferol, propolis, chrysin, apigenin, luteolin, myricetin, acecetin, vitamins including the carotenes and ascorbic acid or with natural light protection filters, such as, for example, rutin, by exploiting a synergistic effect.

Formulations according to the invention, which are a subject-matter of the invention, are characterized by the fact that isoquercitrin is present therein in an amount of from about 0.01 to 10% by weight.

These formulations, which are to be applied to the skin, preferably have a content of light protection filters in the cosmetic preparation of from about 0.01 to 40% by weight of the cosmetic preparation.

Corresponding formulations have a content of isoquercitrin of from about 0.1 to 90% by weight, based on the total amount of light protection filters present in the formulation.

It has been found that the naturally occurring substance isoquercitrin is effective as a UV filter and has an antiviral action against herpes. Isoquercitrin, which is also known under the names isotrifoliin or trifoliin, is present, for example, in a variety of plants, such as, for example, Grosypium herbaceum, Malvaceae, Ribes nigrum (blackcurrants), Aesculuc hippocastanum L. (horse chestnuts, particularly in the flowers), Tropaeolum majus L., Arnica montana L., Corchorus olitorius L., Taxillus levinei, Clematis stans, Hypericum brasiliense and in the pods of the dimorphandra bush. Along with rutin and quercitrin, isoquercitrin belongs to the same aglycone: quercetin. In the intestinal tract, quercetin is produced by hydrolysis of rutin, in some instances via isoquercitrin as an intermediate. Pharmacological investigations (Chanh et al.; Prostaglandins Leukot. Med. 22(3), 295-300 (1986); Kaul et al., J. Med. Virol. 15(1), 71-79 (1985); Amoros et al.; J. Nat. Prod. 55(12), 1732-1740 (1992); Abou-Karam J. Nat. Prod. 55(10), 1525-1527 (1992)) have however shown that these compounds, although belonging to the same aglycone, may have different effects. Whilst quercetin apparently displays virtually no antiviral effects, or only does so in very high doses, this can be unequivocally detected for isoquercitrin. For example, it has been found that the rate of infection by herpes simplex viruses of type I is not influenced by quercitrin, even if the tissue cultures investigated have been cultivated in the presence of quercitrin.

Isoquercitrin in pure form i s a yellow crystalline substance having a melting point of from 225 to 227° C. Experiments have shown that it has a maximum UV light absorption at 257 nm and 359 nm and is thus particularly effective as a UV filter in the UVB wavelength region, although a filter effect can also be detected in the UVA wavelength region. Isoquercitrin can be prepared conventionally. See, e.g., Merk Index, 12th Edition, page 891, No. 5240.

This compound, which occurs in mallow flowers, which may be used for producing tea, and in medicinal plants, such as, for example, arnica or horse chestnuts, is thus highly suitable for incorporation into cosmetic formulations as a UV filter to protect against the damaging effect of UV radiation.

The proven antiviral action against herpes simplex viruses thus advantageously also achieves protection against the formation of herpes blisters, which increase in number in cases of existing infection and with solar irradiation.

Isoquercitrin also has the great advantage of being well tolerated by the skin and thus causes virtually no toxic or allergic reaction.

Isoquercitrin can be incorporated into cosmetic formulations in concentration of up to about 40% by weight of the formulation. It is, as already stated above, a crystalline compound which is readily formulations, if necessary using a solubilizer.

As well as isoquercitrin, the UV protection of the formulations can be improved by adding other natural UV filters, such as, for example, the compounds quercetin or rutin, which belong to the same aglycone and which are known as naturally occurring UV filters.

The formulations according to the invention can also be used for the preventative treatment of inflammation and allergies of the skin, for preventing certain types of cancer and also, if necessary with the addition of suitable active ingredients, for repelling insects.

Under the abovementioned conditions, isoquercitrin can be distributed uniformly in the conventional cosmetic carriers and, when formulated appropriately, in particular in fatty carriers, can form a continuous film; in this way, it is possible to apply them to the skin in order to form an effective protective film.

Formulations which comprise isoquercitrin may if necessary be modified to increase the photostability of other light protection filters present, as described in EP 0 717 982, by adding an effective amount of an amide compound. This may also be amide compounds such as, for example, ethyl 3-(N-butylacetamino)propionate.

The invention thus provides for the use of isoquercitrin in cosmetic formulations.

The invention also provides cosmetic formulations comprising at least one naturally occurring light protection filter such as, for example, rutin.

In a preferred embodiment, as well as isoquercitrin, light protection filters from the group consisting of cinnamic acid esters, such as octyl p-methoxycinnamate (Eusolex® 2292, Merck KGaA), isoamyl p-methoxycinnamate, octyl methoxycinnamate and/or diethanolamine p-methoxycinnamate, are used. Particular preference is given to using Eusolex® 2292.

Formulations comprising isoquercitrin may, as already stated above, also contain light protection filters from the group consisting of the benzophenones, benzoylmethane derivatives and dibenzoylmethane derivatives or UVA filters which are, for example, obtainable commercially under the names Eusolex® 4360, Eusolex® 8020 or Eusolex® 9020. UVA filters and also UVB filters may be present individually or in combinations in formulations according to the invention.

The content of light protection filters in the cosmetic formulations according to the invention is preferably from about 0.01 to 40% by weight, a content of from about 1 to 20% by weight being very particularly preferred. Another very particularly preferred embodiment comprises from 3 to 10% by weight.

The content of isoquercitrin is preferably from about 0.1 to 90% by weight, particularly preferably from about 1 to 30% by weight, based on the total amount of light protection filter present.

The naturally occurring light protection filters, selected from the group consisting of isoquercitrin, rutin, quercetin, quercitrin, catechol and hesperitin, may be present in the cosmetic formulations according to the invention on their own or in combination with one or more UV filters from other classes of substances, which may be present in each case in an amount of from about 0.01 to 40% by weight, preferably from about 0.1 to 30% by weight. In addition, isorhamnetin, kaempferol, glucosides thereof, eriodictyol or substances already mentioned above may be present in appropriate amounts and, in some cases, enhance the effect of the isoquercitrin as a result of synergistic effects.

Since Eusolex® 2292 is, as already mentioned, a very good solubilizer for other UV filters, it can be used combined with other UV filters, as a result of which it is possible to achieve an increased overall concentration of light protection filter present in prepared formulations.

The light protection filters, selected from the group consisting of isoquercitrin, rutin and quercetin, quercitrin, catechol and hesperitin, may be obtained commercially as known natural materials.

Formulations which are obtained using, in particular, isoquercitrin are sunscreens and so as well as their antiviral action have excellent properties, such as very good skin and mucosal tolerability without toxic, allergenic or sensitizing properties.

Isoquercitrin as a substance on its own has high chemical stability, i.e. does not undergo hydrolysis, and has high thermal stability and high perspiration resistance.

Furthermore, this compound is very compatible and miscible with common cosmetic and pharmaceutical formulation bases.

By virtue of its solubility profile, the compound is readily miscible, in some cases using solubilizers, with oils, fats and emulsions and, in view of its profile of properties, can be used in combination with other known light protection filters. It is particularly readily soluble in polar solvents, such as, for example, ethanol or 1,2-propylene glycol. It is soluble in glycerol and forms a slightly cloudy solution. In view of the property profile of isoquercitrin, it is easy for the person skilled in the art to choose suitable solvents and additives depending on the desired preparation product. It is particularly easy to prepare emulsions, gels or emulsion care sticks with isoquercitrin. It is, however, also possible to prepare all other cosmetic formulations, such as ointments, creams, lotions, oils, sticks and sprays, and also pharmaceutical formulations in solid or liquid form using appropriate additives.

A combination with ethyl 3-(N-butyl-acetamino) propionate and Eusolex® 2292 is also suitable for incorporating UV filters which are otherwise soluble only in low concentrations into the cosmetic preparations, which is associated with an increased photostability of the formulation.

If the cosmetic formulation according to the invention is used to protect the human epidermis from UV rays, it is in various forms usually used for this type. Thus, for example, it can in particular be in the form of oily, oily-aqueous, aqueous-alcoholic or oily-alcoholic lotions, emulsions, such as a cream or milk (W/O or O/W), in the form of oily-alcoholic, oily-aqueous or aqueous-alcoholic gels or as solid sticks or powders or formulated as a spray or aerosol.

The formulation according to the invention may contain other cosmetic additives which are usually used in this type of preparation, such as for example thickeners, emollients, humectants, surfactants, preservatives, antifoams, perfumes, waxes, lanolin, propellants, dyes and/or pigments which colour the composition itself or the skin, and other ingredients usually used in cosmetics.

As a solubilizing agent it is possible to use an oil, wax or other fatty substance, a lower monoalcohol or a lower polyol or mixtures thereof. Particularly preferred monoalcohols or polyols include ethanol, i-propanol, propylene glycol, glycerol and sorbitol.

One preferred embodiment of the invention is an emulsion which is in the form of a protective cream or milk and which, in addition to one or more light protection filters and the isoquercitrin, comprises fatty alcohols, fatty acid esters, in particular triglycerides of fatty acids, fatty acids, lanolin, natural or synthetic oils or waxes and emulsifiers in the presence of water.

Other preferred embodiments include oily lotions based on natural or synthetic oils and waxes, lanolin, fatty acid esters, in particular triglycerides of fatty acids, or oily-alcoholic lotions based on a lower alcohol, such as ethanol, or a glycol, such as propylene glycol, and/or a polyol, such as glycerol, and oils, waxes and fatty acid esters, such as triglycerides of fatty acids.

The cosmetic preparation according to the invention can also be in the form of an alcoholic gel which comprises one or more lower alcohols or lower polyols, such as ethanol, propylene glycol or glycerol, and a thickener such as diatomaceous earth. The oily-alcoholic gels also comprise natural or synthetic oil or wax.

A very particularly preferred embodiment consists in solid sticks, such as, for example, in the form of fatty sticks for the lips or sunblocks. These consist of natural or synthetic waxes and oils, fatty alcohols, fatty acids, fatty acid esters, lanolin and other fatty substances, into which the UV filters and the isoquercitrin are incorporated by methods known to the person skilled in the art.

If a preparation is formulated as an aerosol, the customary propellants, such as alkanes, fluoroalkanes and chlorofluoroalkanes, preferably CO₂ or air, are usually used.

If a formulation is formulated as a spray, aqueous-alcoholic solutions are usually used.

As already mentioned above, medicinal formulations for the prevention and treatment of herpes infections or other viral infections, in particular of the skin, are likewise a subject-matter of the present invention.

Isoquercitrin and its physiological acceptable salts can thus be used for the preparation of pharmaceutical preparations by converting them, together with at least one carrier or auxiliary and, if desired, with one or more further active ingredients, into the appropriate dosage form. In particular, it is possible, to exploit a synergistic effect, to use isoquercitrin alone or in combination with other active ingredients.

The resulting preparations can be used as medicaments in human medicine or veterinary medicine. Suitable carriers are organic or inorganic substances which are suitable for enteral (e.g. oral) or parenteral application or for application in the form of an inhalation spray and do not react with the novel compounds, for example water, vegetable oils, benzyl alcohols, polyethylene glycols, glycerol triacetate and other fatty acid glycerides, gelatins, soybean lecithin, carbohydrates, such as lactose or starch, magnesium stearate, talc or cellulose.

For oral use, tablets, dragées, capsules, syrups, juices or drops are used in particular; of particular interest are coated tablets and capsules having enteric coatings or capsule shells.

For parenteral application, solutions, preferably oily or aqueous solutions, and also suspensions, emulsions or implants are used. For application as nasal, mouth or inhalation spray, sprays may be used which contain the active ingredient either in dissolved or suspended form. Particularly in an inhalation spray, it is possible to administer the compound in a propellant mixture. The active ingredient is advantageously used for this purpose in micronized form, in which case it is possible for one or more additional physiologically acceptable solvents, e.g. ethanol, to be present. Inhalation solutions may be administered using customary inhalers.

The active ingredient claimed according to the invention can also be used in lyophilized form and the resulting lyophilizates can be used, for example, to prepare injection preparations. The stated preparations can be sterilized and/or may comprise auxiliaries such as preservatives, stabilizers and/or wetting agents, emulsifiers, salts for influencing the osmotic pressure, buffer substances, dyes and/or aroma substances. They may, if desired, also comprise one or more further active ingredients, e.g. one or more vitamins, diuretics or antiphlogistics.

According to the invention, isoquercitrin is usually administered analogously to other known preparations available commercially, but in particular analogously to the compounds described in U.S. Pat. No. 4,880,804, preferably in dosages between about 1 mg and 1 g, in particular between 25 and 500 mg, per dose unit. The daily dose is preferably between about 0.1 and 50 mg/kg, in particular between 1 and 10 mg/kg of body weight. The specific dose for each individual patient will, however, depend on a wide variety of factors, for example on the effectiveness of the specific active ingredient combination used, age, bodyweight, general state of health, sex, diet, time and route of administration, rate of excretion, medicament combination and the severity of the respective disorder to which the therapy is applied.

Topical application is preferred.

The cosmetic preparations according to the invention can be prepared using techniques well known to the person skilled in the art.

It is assumed that a person skilled in the art will be able to utilize the above description in the widest sense without further explanation. The preferred embodiments are therefore merely to be seen as a descriptive disclosure which is no way limiting

The complete disclosure of all applications, parents and publications given above are incorporated into this application by reference in their entirety.

The examples below serve to explain the invention in more detail.

### EXAMPLES

### Example 1

Lip balm containing isoquercitrin

| | | % by |
|---|---|---|
| | | weight |
| A | | |
| Isoquercitrin | (1) | 0.1 |
| Cremophor A 25 | (2) Ceteareth-25 | 20.0 |
| Cetiol HE | (3) PEG-7 Glyceryl Cocoate | 22.0 |
| B | | 5.0 |
| Glycerol | Glycerin | q.s. |
| Preservative | Aqua | ad 100 |
| Demin. water | | |

Preparation

Phases A and B are heated to a temperature of 800C. B is added to A with stirring and the mixture is cooled to room temperature to give a colourless gel.

Suppliers:

(1) Merck KGaA, Darmstadt

(2) BASF

(3) Henkel KGaA, Düsseldorf

1. A pharmaceutical or cosmetic composition comprising an amount of isoquercitrin and a carrier.

2. A composition of claim 1, which is a pharmaceutical composition and comprises an amount of isoquercitrin which is effective as an antiviral agent.

3. A composition of claim 2, wherein said antiviral agent is effective against Herpesvirdae.

4. A composition of claim 3, which is effective against herpes simplex virus.

5. A composition of claim 1, which is a cosmetic composition for topical application and comprises an amount of isoquercitrin which is effective as a UV filter.

6. A composition of claim 5, wherein said amount of isoquercitrin is effective as an antiviral agent.

7. A composition of claim 1, wherein said composition is ointment, cream, milk, lotion, emulsion, oil, gel, or liquid spray for topical application to the skin.

8. A method of claim 1, wherein said composition is a tablet, dragee, capsule, syrup, juice, or drops.

9. A composition of claim 1 for topical application to the skin, further comprising a UV-A or UV-B filter.

10. A composition of claim 9, where said UV filtering agent is benzophenones, benzoylmethane derivatives and dibenzoylmethane derivatives, cinnamic acid esters, isoamyl methoxycinnamate, octyl methoxycinnamate, triazines, benzylidenecamphor, PABA, salicylates, octocrylene, ZnO or TiO₂.

11. A composition of claim 1, comprising synergistically-effective amounts of said isoquercitrin and synergistically-effective amounts of 5-ethyldeoxyuridine, quercetin, galangin, kaempferol, propolis, chrysin, apigenin, luteolin, myricetin, acacetin, eriodictyol, isorhamnetin, or a glycoside thereof, vitamins, carotenes, ascorbic acid, or a light protection filter which is quercetin, quercitrin, catechol, hesperitin, rutin, or a glycoside thereof.

12. A composition of claim 1, wherein said isoquercitrin is present in an amount of 0.01-10% by weight of said composition.

13. A composition of claim 1, further comprising a light protection filter, wherein said light protection filter is present in an amount of 0.01-40% by weight of said composition.

14. A composition of claim 1, further comprising an amount of a light protection filter, wherein said isoquercitrin is present in an amount of 0.1-90% by weight based on the total amount of light protection filter in said composition.

15. A topical composition for application to the skin, comprising an amount of isoquercitrin which is effective for UV light protection.
